(19) United States
(12) Patent Application Publication
(10) Pub. No.: US 2026/0035747 A1
(43) Pub. Date: Feb. 5, 2026

(54) TISSUE BASED RNA IN SITU HYBRIDIZATION FOR DIAGNOSIS AND TREATMENT SELECTION IN DERMATOLOGY

(71) Applicant: Yale University, New Haven, CT (US)

(72) Inventor: William Damsky, New Haven, CT (US)

(21) Appl. No.: 19/298,740

(22) Filed: Aug. 13, 2025

Related U.S. Application Data
(63) Continuation of application No. 17/471,972, filed on Sep. 10, 2021, now abandoned.
(60) Provisional application No. 63/077,143, filed on Sep. 11, 2020.
(60) Provisional application No. 63/185,930, filed on May 7, 2021.

(51) Int. Cl. C12Q 1/6883; C07K 16/24; C12Q 1/6841

(52) U.S. Cl. CPC C12Q 1/6883; C07K 16/244; C12Q 1/6841; C12Q 2600/158

(57) ABSTRACT

In various aspects and embodiments the invention provides a method of treating an autoimmune or autoinflammatory dermatological condition in a subject in need thereof, the method comprising: performing RNA in situ hybridization on a tissue sample obtained from the patient with at least one probe that binds to a polyribonucleotide encoding at least one cytokine to determine a level of the at least one cytokine; comparing the level of the at least one cytokine to a normal control; determining that the level of the at least one cytokine deviates from the normal control; and providing treatment for the dermatological condition to the subject.

### CROSS-REFERENCE TO RELATED APPLICATIONS

This application is a continuation of U.S. patent application Ser. No. 17/471,972, filed Sep. 10, 2021, which claims priority under 35 U.S.C. § 119 (e) to U.S. Provisional Patent Application No. 63/077,143 filed Sep. 11, 2020 and U.S. Provisional Patent Application No. 63/185,930 filed May 7, 2021, each of which is incorporated herein by reference in its entirety.

### BACKGROUND OF THE INVENTION

In dermatology, monoclonal antibodies that block specific cytokines are commonly used in the treatment of inflammatory skin disorders such as psoriasis and atopic dermatitis. More and more agents targeting different cytokines are becoming available; with multiple being approved for the same disease. For example, in psoriasis, TNF-alpha, IL-12/23, IL-17A, IL-17R (IL-17A/F), and IL-23 inhibitors are all available and more are coming. Selection of a specific agent for a particular patient is currently based on physician preference, insurance coverage, and other factors; but immunologic criteria that might be unique to each patient are not incorporated into the decision making process. This results in treatment by trial-and-error and has the implications of exposure of patients to unnecessary side effects and potentially a patient remaining on a therapy to which they have a suboptimal response. There is a need in the art for an easy to implement means to evaluate diagnostic biopsies for expression (or not) of cytokines targeted by a particular drug to inform treatment selection. The present disclosure addresses this need.

### SUMMARY OF THE INVENTION

In one aspect, the invention provides a method of treating an autoimmune or autoinflammatory dermatological condition in a subject in need thereof, the method comprising:
performing RNA in situ hybridization on a tissue sample obtained from the patient with at least one probe that binds to a polyribonucleotide encoding at least one cytokine to determine a level of the at least one cytokine;
comparing the level of the at least one cytokine to the level of the same cytokine in a normal control;
determining that the level of the at least one cytokine deviates from the level of the same cytokine in the normal control; and
providing treatment for the dermatological condition to the subject.

In various embodiments, the tissue sample is a formalin-fixed paraffin-embedded tissue sample.

In various embodiments, the tissue sample is a diagnostic biopsy sample.

In various embodiments, the cytokine is selected from the group consisting of IL12B, IL17A, IL17C, IL17F, IL23A, IL4 and IL13.

In various embodiments, the probe binds IL-12 p40, IL-17A, IL-23 p19, IL-4 or IL-13.

In various embodiments, the cytokine is selected from the group consisting of IL31, IL33, TSLP, IL12A, IL22, IL36A and IL36G.

In various embodiments, the method further comprises measuring a level of NOS2.

In various embodiments, the treatment comprises a monoclonal blocking antibody targeting the at least one cytokine.

In various embodiments, the dermatological condition is psoriasis and the treatment is ustekinumab.

In various embodiments, the dermatological condition is atopic dermatitis and the treatment is dupilumab.

In various embodiments, the RNA in situ hybridization comprises RNAscope analysis.

In various embodiments, the at least one cytokine comprises a panel of three to five cytokines.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIGS. 1A-1B: IL-12B, IL-17A, IL-23A, IL-13, and IL-4 RNA ISH staining of cases of psoriasis, dermatitis, and normal skin. FIG. 1A: Representative staining patterns for each target in each condition, 600×magnification. FIG. 1B: Violin plots showing mean and standard deviation (S.D) for each condition. *p<0.05, **p<0.001 ***p<0.0001, NS: not significant.

FIGS. 2A-2B: IL-13 and IL-17A RNA ISH staining patterns in psoriasis, dermatitis, and intermediate cases. FIG. 2A: IL-17A versus IL-13 staining for each individual case of psoriasis and dermatitis. Differently shaded areas represent a psoriasis (marked “b”) versus a dermatitis pattern (marked “b”), respectively. Inset showing percentage of psoriasis and dermatitis cases with each pattern of cytokine expression: double negative (IL13−IL17A−), psoriasis pattern (IL13−IL17A+, shaded box), dermatitis pattern (IL13+IL17A−), and double negative. FIG. 2B: IL-17A versus IL-13 staining for each intermediate case. Inset as in FIG. 2A.

FIGS. 3A: Example of IL-17A staining of intra-epidermal lymphocytes in psoriasis. FIG. 3B: Example of representative IL-13 staining of intra-epidermal lymphocytes in dermatitis.

FIG. 4A: Example of representative IL-12 p40 staining of clusters of intra-epidermal cells in psoriasis. FIG. 4B: Example of representative IL-12 p40 staining of occasional single intra-epidermal cells in dermatitis.

FIG. 5A: Example of representative IL-23 p19 staining of clusters of intra-epidermal cells in psoriasis. FIG. 5B: Example of representative IL-23 p19 staining of rare single intra-epidermal cells in dermatitis.

FIGS. 6A-6B: Cytokine expression patterns differentiate psoriasis and AD, but heterogeneity exists. FIG. 6A: Volcano plot showing relative mRNA expression of selected targets in cases of psoriasis (n=28), AD (n=27), and normal skin from healthy controls (n=38) based on RNA-seq data analysis. FIG. 6B: mRNA expression levels of selected targets among individual patients with each diagnosis, corresponding to data in (a).

FIGS. 7A-7C: NOS2 RISH staining differentiates psoriasis from AD and normal skin. FIG. 7A: Hematoxylin and eosin (H&E) stained sections (left panels), NOS2 RISH staining (middle panels), and quantitative analysis (right panels). Representative cases are shown for each psoriasis, AD, and normal controls, 100×. FIG. 7B: NOS2 RISH staining in a representative case of psoriasis, 600×. FIG. 7C: Quantification of NOS2 RISH in psoriasis, AD, and normal skin (Norm), shown as number of positive cells per millimeter (mm) of epidermis.

FIGS. 8A-8C: IL17A and IL13 RISH staining patterns differentiate psoriasis from AD and normal skin. FIG. 8A: IL17A RISH staining at 200×magnification (left panels) and 600×magnification (middle panels) and quantitative analysis at 600×magnification (right panels). Representative cases are shown for each psoriasis, AD, and normal controls. FIG. 8B: Quantification of IL17A RISH staining data in psoriasis, AD, and normal controls. The number of positive cells in epidermis (left) and dermis (right) were quantified independently. FIG. 8C: IL13 RISH staining at 200×magnification (left panels) and 600×magnification (middle panels) and quantitative analysis at 600×magnification (right panels). Representative cases are shown for each psoriasis, AD, and normal controls. FIG. 8D: Quantification of IL13 RISH staining data in psoriasis, AD, and normal controls (Norm); number of positive cells in epidermis (left) and dermis (right) quantified independently.

FIGS. 9A-9D: IL13 and IL17A expression localizes to CD3+T lymphocytes. FIG. 9A: Double stain showing IL13 RISH (red) and CD3 immunohistochemistry (IHC) (brown) in a representative case of AD (600×magnification). Representative analysis using QuPath and Image J are shown. FIG. 9B: Quantification of the number of IL13 positive cells as a function of total CD3+ cells in AD (n=7). FIG. 9C: Double stain showing IL17A ISH (red) and CD3 IHC (brown) in a representative case of psoriasis (600×magnification). Representative analysis using QuPath and Image J are shown. FIG. 9D: Quantification of the number of IL17A positive cells as a function of total CD3+ cells in psoriasis (n=7).

FIGS. 10A-10D: IL17F staining correlates with IL17A staining in psoriasis. FIG. 10A: IL17F RISH staining (left panel) and quantitative analysis (right panel) in a representative case of psoriasis, 600×. FIG. 10B: Quantification of IL17A and IL17F RISH staining. The number of positive cells in the epidermis (left) and dermis (right) were quantified independently. FIG. 10C: Scatter plot of IL17A vs IL17F levels in the epidermis of psoriasis and AD cases. FIG. 10D: Scatter plot of IL17A vs IL13 levels in the epidermis of psoriasis and AD cases.

FIGS. 11A-11D: IL4, IL12B, AND IL23A staining can also help classify cases of psoriasis and AD. FIG. 11A: IL4 RISH staining at 200×magnification (left panels) and 600×magnification (middle panels) and quantitative analysis at 600×magnification (right panels). Representative cases are shown for each psoriasis, AD, and normal controls. FIG. 11B: IL12B RISH staining at 200×magnification (left panels) and 600×magnification (middle panels) and quantitative analysis at 600×magnification (right panels). Representative cases are shown for each psoriasis, AD, and normal controls. FIG. 11C: IL23A RISH staining at 200×magnification (left panels) and 600×magnification (middle panels) and quantitative analysis at 600×magnification (right panels). Representative cases are shown for each psoriasis, AD, and normal controls. FIG. 11D: Quantification of IL4, IL12B, IL23A RISH in psoriasis, AD, and normal skin (Norm), shown as number of positive cells per millimeter (mm) of epidermis (left panels) or dermis (right panels).

FIGS. 12A-12C: IL22, IL31, IFNG, and TNF staining patterns in psoriasis and AD

FIG. 12A: Representative IL23A and IL12B staining patterns in psoriasis. FIG. 12B: Representative IL23A and IL12B staining patterns in AD. FIG. 12C: Quantification of IFNG, IL22, IL31, and TNF RISH in psoriasis, AD, and normal skin (Norm), shown as number of positive cells per millimeter (mm) of epidermis (upper panels) or dermis (lower panels).

FIGS. 13A-13F: Analysis of scRNA-seq data in psoriasis (n=3), AD (n=4), and control skin (n=5). Re-analysis of data from Reynolds et al. (Reynolds et al., 2021). FIG. 13A: t-SNE plot of keratinocytes (KC), myeloid cells, and T cells colored by library type. FIG. 13B: t-SNE in (a) colored by epidermal-location vs dermal-location. FIG. 13C: Bar graphs showing the proportion of different T cell populations in the epidermis and dermis. FIGS. 13A-13F: Dot plots showing cytokine expression by various cell types. For FIGS. 13C-13F: cell identity based on designations by Reynolds et al.

FIGS. 14A-14C: Psoriasis and AD cases can be classified based on cytokine expression patterns determined by RISH. FIG. 14A: Principal component analysis (PCA) of psoriasis, AD and normal control based on RISH staining pattern for IL17A, IL17F, IL12B, IL23A, NOS2, IL13, and IL4 in the epidermis. FIG. 14B: Biplot of the PCA data for psoriasis drivers. (c) Biplot of the PCA data for AD drivers.

### DETAILED DESCRIPTION

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The “level” of one or more molecules of interest, for example a cytokine, means the absolute or relative amount or concentration of the molecule in the sample as determined by measuring mRNA, cDNA or protein, or any portion thereof such as oligonucleotide or peptide.

“RNAScope” as used herein refers to RNA in situ hybridization using a plurality of probes having a double-Z design as described in Wang et al., RNAScope, J Mol Diagn. 2012; 14(1): 22-29.

“Tissue sample” as used herein refers to a sample of solid cells from a patient, such as from a biopsy.

As used herein, “treating a disease or disorder” means reducing the frequency with which a symptom of the disease or disorder is experienced by a patient. Disease and disorder are used interchangeably herein.

As used herein, the term “treatment” or “treating” encompasses prophylaxis and/or therapy. Accordingly the compositions and methods of the present invention are not limited to therapeutic applications and can be used in prophylactic ones. Therefore “treating” or “treatment” of a state, disorder or condition includes: (i) preventing or delaying the appearance of clinical symptoms of the state, disorder or condition developing in a subject that may be afflicted with or predisposed to the state, disorder or condition but does not yet experience or display clinical or subclinical symptoms of the state, disorder or condition, (ii) inhibiting the state, disorder or condition, i.e., arresting or reducing the development of the disease or at least one clinical or subclinical symptom thereof, or (iii) relieving the disease, i.e. causing regression of the state, disorder or condition or at least one of its clinical or subclinical symptoms.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

In one aspect, the invention provides a method of treating an autoimmune or autoinflammatory dermatological condition in a subject in need thereof, the method comprising performing RNA in situ hybridization on a tissue sample obtained from the patient with at least one probe that binds a polyribonucleotide encoding at least one cytokine to determine a level of the at least one cytokine; comparing the level of the at least one cytokine to the level of the same cytokine in a normal control; determining that the level of the at least one cytokine deviates from the level of the same cytokine in the normal control; and providing treatment for the dermatological condition to the subject. In various embodiments, the invention is based in part on employing RNA in situ hybridization in diagnostic skin biopsies to detect specific cytokine targets of currently available targeted inhibitors for psoriasis and atopic dermatitis. Accordingly, in various embodiments, the tissue sample is a formalin-fixed paraffin-embedded tissue sample. In various embodiments, the tissue sample is a diagnostic biopsy sample.

As shown in the Examples, this method accurately classified each case as psoriasis versus dermatitis based on cytokine expression profiles. There was significant heterogeneity in terms of which cytokines were expressed and at what relative level by this method in each individual case. Selecting a targeted therapy directed against the cytokine(s) which are most highly expressed in an individual patients biopsy produces a more optimal response (as opposed to the current trial and error approach). In various embodiments, the cytokine is selected from the group consisting of IL12B, IL17A, IL17C, IL17F, IL23A, IL4 and IL13. In various embodiments, the probe binds a polyribonucleotide encoding IL-12 p40, IL-17A, IL17C, IL17F, IL-23 p19, IL-4 or IL-13. In various embodiments, the treatment comprises a monoclonal blocking antibody targeting the at least one cytokine. Without meaning to be limited by theory, in various embodiments, when a specific cytokine is upregulated in a tissue sample as part of an autoimmune or autoinflammatory condition in a subject, the subject will respond to treatment with an antibody that binds and blocks the activity of the cytokine. In various embodiments, the treatment is an inhibitor that targets IL-17A, IL17C, IL17F, IL-17R, IL23, IL4 or IL13 In various embodiments, the treatment is selected from the group consisting of secukinumab, ixekizumab, brodalumab, guselkumab, tildrakizumab, risankizumab, Mirikizumab, Bimekizumab, tralokinumab, lebrikizumab, ustekinumab and dupilumab. In various embodiments, the dermatological condition is psoriasis and the treatment is ustekinumab. In various embodiments, the dermatological condition is atopic dermatitis and the treatment is dupilumab. In various embodiments, the condition to be treated, the elevated cytokine and the treatment targeting that cytokine are selected from:

### Psoriasis

Ustekinumab—IL-12/23p40
Secukinumab—IL-17A
Ixekizumab—IL-17A
Brodalumab—IL-17receptor
Guselkumab—IL-23A
Risankizumab—IL-23A
Tildrakizumab—IL-23A
Mirikizumab—IL-23A (clinical trials)
Bimekizumab—IL17A/F (clinical trials)
Deucravacitinib-JAK (TYK2) inhibitor

### Atopic Dermatitis

Dupilumab—IL4/IL13
Lebrikizumab—IL13 specific (clinical trials)
Traolkinumab—IL13 specific (clinical trials)
Abrocitinib—JAK inhibitor (clinical trials)
Baricitinib—JAK inhibitor
Ruxolitinib—JAK inhibitor
Tofacitinib—JAK inhibitor
Upadacitinib—JAK inhibitor

In various embodiments, the cytokine is selected from the group consisting of IL31, IL33, TSLP, IL12A, IL22, IL36A and IL36G. In various embodiments, the method further comprises measuring a level of NOS2. NOS2 is the gold standard in differentiating Th17 polarization (psoriasis, positive) from Th2 polarization (AD, negative).

In various embodiments, the RNA in situ hybridization comprises RNAscope analysis. Probes and materials for RNAScope are available commercially or may be generated by persons of ordinary skill in the art.

### Experimental Examples

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

The materials and methods employed in practicing the following examples are here described:

### Histologic Case Series

Cases were selected by searching the Yale Dermatopathology database for archival material. This work was reviewed and approved by the Yale Institutional Review Board. Cases were selected randomly in the archive if they met criteria to fit in one of three groups: classic psoriasis, classic dermatitis, and intermediate. Classic psoriasis cases had classic features of psoriasis (a top line diagnosis of psoriasis was rendered) on the biopsy and the clinical impression favored psoriasis. Classic dermatitis cases showed spongiotic dermatitis on the biopsy and the clinical impression favored dermatitis. For intermediate cases, features of both psoriasis and dermatitis were present in the specimen precluding definitive diagnosis and the clinical impression at the time of the biopsy included both psoriasis versus dermatitis. In general biopsies used in this study, including the dermatitis cases, were from adults.

### RNA In Situ Hybridization

RNA in situ hybridization (ISH) was performed using the RNAscope kit (Bio-techne) following the manufacturer's instructions. Briefly, slides were deparaffinized and then hydrogen peroxidase and protease activity were blocked by using hydrogen peroxide and protease plus from the RNA scope kit (Cat #322330). Antigen retrieval was performed with ACDbio target retrieval reagent (Cat #322000). Probe hybridization and amplification steps were performed according to the manufacturer's instructions. Slides were counterstained with hematoxylin and cover slipped. Probes for IL17A (Cat #310931), IL12B (IL-12 p40) (Cat #402071), IL23A (IL-23 p19) (Cat #562851), IL4 (Cat #315191), and IL/3 (Cat #586241) were purchases from Bio-techne.

### Quantification of Staining

The number of positive cells in each specimen was quantified manually. A separate count was obtained for the epidermal and dermal portions. The number of positive cells was divided by the width of the biopsy in millimeters to correct for differences in biopsy size (cells/mm). In order for a cell to be counted as positive, staining needed to be clearly apparent with the 20× objective and at least two dots per cell had to be present (most positive cells had >>2 dots). Faint single dots apparent only with the 40× objective were not included as positive (these cells were quite rare). For the quantification, any specimens less than 3 mm in width without any positive staining were excluded from the analysis. These samples were excluded because we noticed in larger specimens that staining was patchy. The slides were scored in a blinded fashion by consensus. Although the diagnosis was not considered during the scoring; morphologie features in some cases could not be entirely ignored.

For singly stained slides, the images were imported into QuPath. A region of interest that covered the area of interest was selected. Stain vectors were defined by selecting a region of interest (ROI) that was representative of the stain. The positive cell detection feature was utilized to identify positive cells for each marker. The automated detection was manually verified and, in some cases, adjustments were made manually. To facilitate visualization, cells were colored based on whether or not they were positive for the marker.

For double stained slides, the images were imported into QuPath and the color vectors were defined using ROI as described above. The he_heavy_augement model, a brightfield image nucleus StarDist (arXiv: 1806.03535) approach, was then used in QuPath to detect cell nuclei. After nucleus detection using this approach, the images were then imported into Fiji for color deconvolution based on the stain vectors defined in QuPath. Color deconvolution allowed separation of the different channels from each stain. Brightness and contrast for each channel was then adjusted (this was done manually) to maximize signal to background ratio using Fiji. The deconvoluted, optimized images were then re-overlaid using Photoshop (Version 21.2.0).

### RNA-Seq Analysis

A previously published bulk RNA-seq dataset was utilized (Tsoi et al., 2019). Read counts were downloaded from Gene Expression Omnibus (GSE121212). Data analysis was performed using Partek Flow software (Version 9.0, build 9.0.20.0510). AD and psoriasis lesional skin and healthy control samples were extracted from the rest of the data set (which also included non-lesional skin). Read counts for these samples were extracted to generate bar graphs using tidyr (Version 1.1.0), ggplot2 (Version 3.3.2) and data.table (Version 1.12.8). The entire dataset was normalized in Partek flow. DESeq2 was used for differential gene expression analysis to compare psoriasis and AD samples in the data set. The Volcano plot was generated using dplyr (Version 1.0.0), ggplot2 (Version 3.3.2) and ggrepel (Version 0.8.2). The PCA plot was generated by Factoextra (1.0.7) and FactoMineR (2.3) for the manually quantified results.

scRNA-Seq Analysis

A recently published single cell RNA sequencing (scRNA-seq) data set comprised of AD, psoriasis and healthy control skin samples (Reynolds et al., 2021) was analyzed to examine patterns of cytokine expression. In this study, the epidermal and dermal portions of the tissue were separated and then dissociated, analyzed separately. The data from this study are freely available and we downloaded from: https://zenodo.org/record/4569496 #.YE9kGLRKi-V. We focused our analysis of these data on lesional AD, psoriasis and healthy control libraries which were re-analyzed in Seurat (v3.2.0). Non-lesional libraries were not included in our analyses. T cells, myeloid cells and keratinocytes were subsetted based on the cell-type assignments from the original publication, as determined by AutoGeneS, and then re-analyzed. We retained these cell type identities for the subsetting, for certain analyses groups were combined, e.g. T cells (Tc, Tc17_Th17, Tc_IL13_IL22, Th, Treg), ILCs (ILC1_NK, ILC2, ILC3), Macrophages (Inf_Mac, Macro_1, Macro_2, Mono_Mac), DCs (DC1, DC2, MigDC, moDC_1, moDC_2, moDC_3), LC (LC_1, LC_2, LC_3, LC_4), and keratinocytes (Differentiated, Proliferating KC, Undifferentiated KC). In total, we included 127,219 cells in our analysis of the data. The NormalizeData command with a scale factor of 10,000 was used to normalize counts. The data was then further scaled based on the number of transcripts and center gene expression values. Cells were clustered using FindNeighbors and FindClusters commands. The data was visualized by performing t-distributed stochastic neighbor embedding (tSNE). The FeaturePlot command was used to generate gene specific tSNE plots and DotPlot command was used to generate dot plots.

### Statistical Analysis

Statistical analysis was performed using Graphpad Prism 8. P values were calculated using a Student's t-test. For the Chi-squared analysis, a Fisher's exact test was used to compute the p value.

### Example 1

Psoriasis and dermatitis (including atopic, nummular, and others) are common inflammatory skin diseases that are increasingly treated with monoclonal blocking antibodies (‘biologics’). Psoriasis is thought to be a disease primarily driven by T helper 17 (Th17) cells. In psoriasis, IL-23 production by myeloid and other cells promotes activation of Th17 cells which produce IL-17, an effector molecule driving the disease. The role of these cytokines is perhaps best illustrated by efficacy of IL-17A, IL-17 receptor (IL-17RA), and IL-23 (both p19 and p40) inhibitors in treating psoriasis. The p40 subunit is shared with IL-12 and inhibited by ustekinumab, while p40 inhibitors are IL-23 specific. In contrast, dermatitis refers to a group of disorders thought to be primarily driven a T helper 2 (Th2) polarized immune response, with cytokines such as IL-4 and IL-13 appearing to play a central role in many cases. Dupilumab, an IL-4Rx blocking antibody that inhibits the activity of both IL-4 and IL-13 has revolutionized atopic dermatitis treatment and reinforces the central role of these cytokines in this disease.

Although most cases of psoriasis and dermatitis can be readily distinguished from each other based on clinical and/or histologic grounds; others cannot and can represent a diagnostic and treatment challenge. Having immunologic assays that can inform diagnosis and even treatment selection in such patients would be beneficial. Further, even in patients with a straightforward diagnosis, satisfactory responses to targeted therapy is not guaranteed. In psoriasis, approximately 25% of patients do not achieve 90% or greater clearance with IL-17 or IL-23 blockade, while in atopic dermatitis, approximately 70% of patients do not achieve 90% or greater clearance with IL-4Ra blockade. One potential explanation for this observation is that molecular immunologic heterogeneity exists within each diagnostic group and implies drugs targeting particular cytokines might be more or less effective for individual patients.

While it has been possible to address this molecular variability in the laboratory; it has been difficult to effectively apply molecular diagnostics to routine clinical dermatologic practice. This has largely been hindered by the lack of reliable markers that can be used in typical formalin-fixed paraffin-embedded biopsies. Immunohistochemistry for cytokines and/or for markers of Th2 vs Th17 cells (for example) has been attempted, but due to high background staining and other technical issues, has not been widely applied.

In order to address this, we employed an RNA in situ hybridization (ISH) approach to immunophenotype historical, biopsies of psoriasis (n=25) and dermatitis (n=25) from the Yale Dermatopathology archive. 13 cases of normal skin were also included. In particular, each case was stained using a probe for: IL12B (IL-12 p40), IL17A (IL-17A), IL23A (IL-23 p19), IL4 (IL-4) and IL13 (IL-13). These cytokines were selected given their established role in the pathogenesis of each disorder and because they are targets of current antibody-based treatments for psoriasis (IL-12, IL17A, and IL23) and atopic dermatitis (IL-4, IL-13).

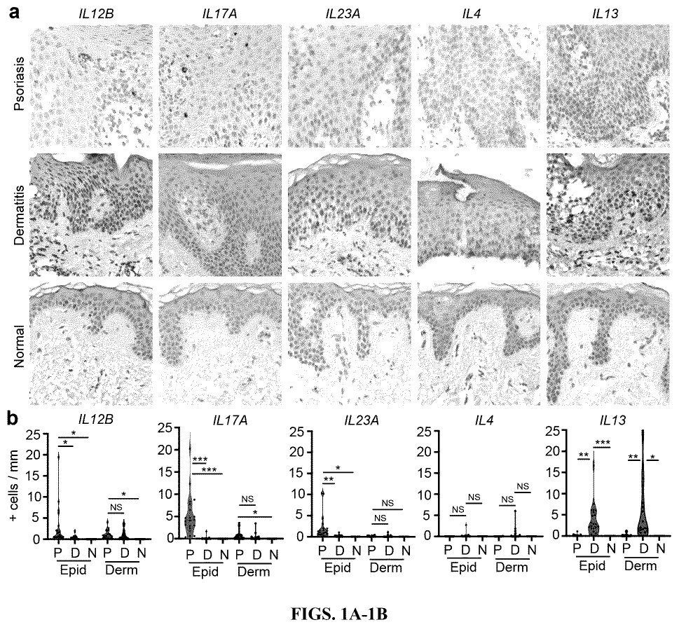

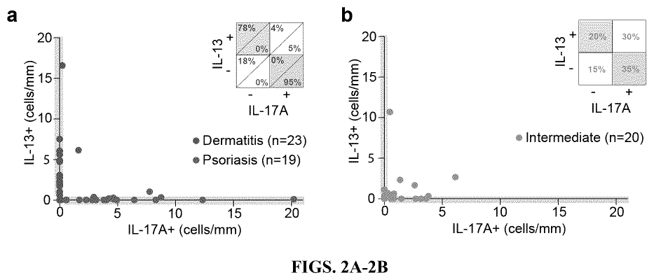

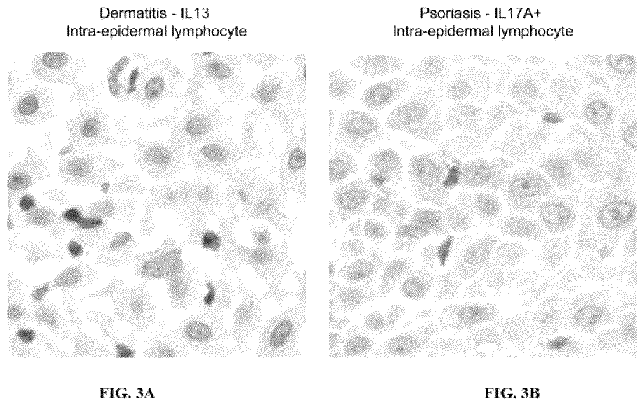

The dermatitis cases stained strongly for IL-13 with most cases exhibiting at least some degree of positivity in both the epidermis and dermis (FIGS. 1A-1B and FIG. 2A). IL-4 expression was much lower than IL-13; and in most cases was undetectable (FIGS. 1A-1B). IL-13 predominance over IL-4 in atopic dermatitis has been reported previously based on RNA-sequencing data and our results are consistent with this observation. IL-13 expressing cells tended to have a morphology consistent with lymphocytes and were most abundant either within or just beneath the epidermis (FIG. 1A, FIG. 3A). Staining for IL-13 was absent in most cases of psoriasis and entirely negative in normal skin (FIGS. 1A-1B). When present in psoriasis, there was only focal, weak positivity and the immunophenotype was otherwise dominated by IL-17A (FIGS. 1A-1B and FIG. 2A). Based on these patterns of staining (FIGS. 3A-3B), we hypothesize that IL-13 ISH is specifically highlighting pathogenic Th2 polarized T cells.

We found that the psoriasis cases stained strongly for IL-17A with all cases exhibiting some degree of positivity (FIGS. 1A-1B and FIG. 2). Staining predominated in cells with the morphology of lymphocytes and tended to be most abundant within the epidermis and much less so in the dermis. There was essentially no staining for IL-17A in the dermatitis cases (except for one case) and none in the normal controls. Interestingly, although IL-17A inhibition has not been widely effective in atopic dermatitis; in a subset of patients IL-17A may play a more prominent role. Based on these patterns of staining (FIGS. 3A-3B), we hypothesize that IL-17A ISH highlights the pathogenic TH17 cells present in psoriasis.

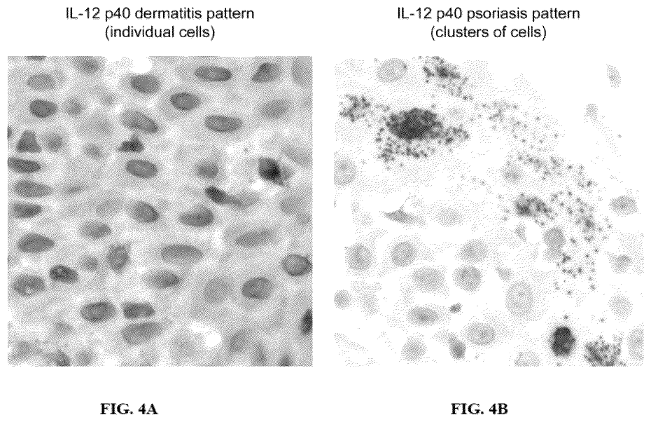

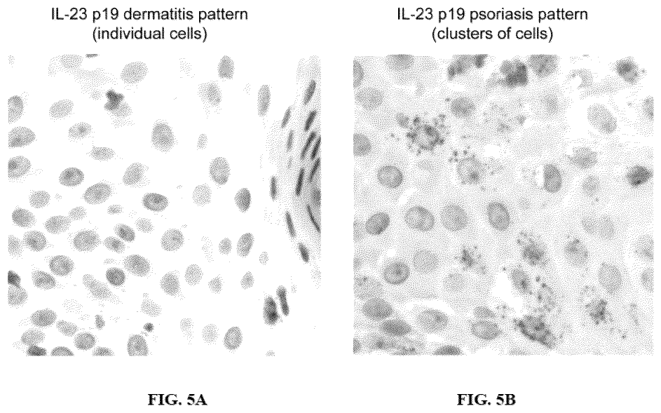

IL-12 (p40) staining within the epidermis was significantly higher in psoriasis compared to dermatitis cases; however, particularly in the dermis, there was significant overlap between the two conditions (FIGS. 1A-1B). IL-23 (p19) staining within the epidermis was present in most of the psoriasis cases and rarely present in dermatitis cases (FIGS. 1A-1B). Very little staining for IL-23 was present in the dermis of both conditions. No staining for either IL-12 or IL-23 was observed in the normal controls. Interestingly, the predominance of the staining for both IL-12 and IL-23 was within the epidermis. In psoriasis, clusters of positive cells predominated; whereas in dermatitis only individual positive cells were present (FIGS. 4A-4B and 5A-5B). In both conditions there were occasional isolated positive cells with a myeloid morphology in the dermis. Although, in psoriasis IL-12 and IL-23 are thought to predominantly be produced by dendritic cells (DCs) in the dermis; production by DCs that either enter the epidermis or reside in the epidermis (i.e. Langerhan's cells) in psoriasis has also been reported previously. Based on the staining patterns observed with our assay; it appears most of the IL-12 and IL-23 production occurs within the epidermis.

Given the ability of IL-17A and IL-13 ISH to distinguish clinicopathologically straightforward cases of psoriasis (IL-17A predominant) and dermatitis (IL-13 predominant) (FIG. 2A), we next evaluated a series of “intermediate” cases with overlapping clinical and histologic features (n=21); that is cases with a clinical differential diagnosis of psoriasis versus dermatitis and with overlapping features histologically precluding definitive diagnosis of either disorder. Interestingly, we observed 4 patterns of staining in this group of cases: 1) double positive, 2) psoriasis-like (IL-17A predominant), 3) dermatitis-like (IL-13 predominant), and 4) double negatives (FIG. 2B). Interestingly, double positive cases were more abundant in the intermediate group than would be expected based on the results of the psoriasis and dermatitis groups (p=0.0095), suggesting that some morphologically intermediate cases are also immunologically overlapping. Other cases could be classified as either psoriasis-like or dermatitis-like; an observation which could have treatment implications.

In summary, we find RNA ISH for cytokines to be a highly informative approach in dermatologic biopsies and demonstrate the utility of this approach in immunophenotyping psoriasis and atopic dermatitis cases, based on IL-17A and IL-13 staining patterns. This approach can be readily expanded to additional cytokine targets and further applied to these and other inflammatory skin diseases.

### Example 2

### Cytokine Expression Patterns Differentiate Psoriasis and AD, but Heterogeneity Exists

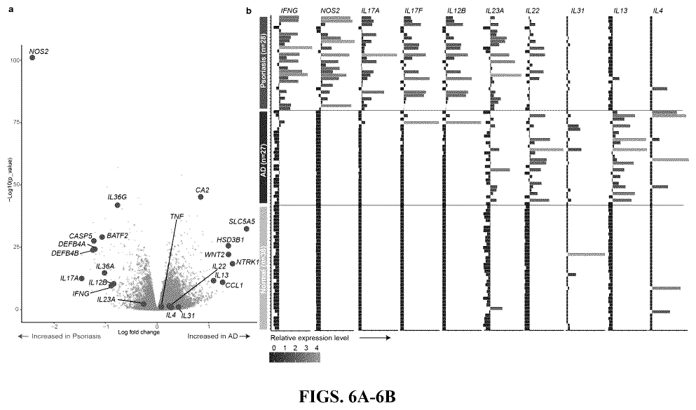

To broadly understand which cytokines and other markers best differentiated between psoriasis and AD lesions, we analyzed bulk RNA-seq data from lesional skin in a cohort of patients with psoriasis (n=28), AD (n=27), and healthy controls (n=38). We found that NOS2 (encodes iNOS) was markedly upregulated in psoriasis and was the most significantly differentially expressed transcript between the two conditions (FIG. 6A). NOS2 upregulation in psoriasis has been observed previously.

We also observed that genes encoding cytokine targets of approved treatments were among the most differentially expressed transcripts. In particular, IL17A and IL13 were markedly upregulated in psoriasis and AD, respectively. IL12B (encodes IL-12/23 p40), IL23A (encodes IL-23 p19), and IL17F were also upregulated in psoriasis, as expected. Significant IL4 expression was not detected in these samples, although did tend to be higher in AD, consistent with prior reports. Emerging treatment targets including IL31 and IL22, as well as IL36A/G were increased in AD and psoriasis, respectively.

Next, we assessed the heterogeneity in expression of key cytokines within these samples. We found that there was considerable variability within both psoriasis and AD in terms of the predominant druggable cytokine expressed (FIG. 6B). For example, some cases of psoriasis were IL17A-predominant, but others expressed very little IL17A, and were instead IL17F, IL23A, and/or IL12B predominant. Interestingly, some cases of AD did not show significant expression of either IL13 or IL4 and one case was IL17F predominant. These data suggest that there is significant molecular heterogeneity among cases of psoriasis and AD, which might have important implications for optimal treatment selection.

iNOS (NOS2) Staining Differentiates Psoriasis from AD

To assess RISH staining in this setting, we assembled cohorts of additional psoriasis (n=20) and AD (n=26) patients in which biopsy tissue was available for study (Table 1).

TABLE 1
Patient characteristics.

| | | | Atopic | | | |
|---|---|---|---|---|---|---|
| Characteristics | Psoriasis | | Dermatitis | | Normal | |
| Number of patients | 20 | | 26 | | 10 | |
| Age in years, | 48, | | 57, | | 50, | |
| median (range) | (18-79) | | (24-83) | | (30-72) | |
| Sex, male (%) | 70% M; | | 68% M; | | 23% M; | |
| female(%) | 30% F | | 32% F | | 77% F | |
| Anatomic site, | | | | | | |
| n/total (%) | | | | | | |
| Trunk | 7/20 | (35%) | 7/26 | (27%) | 5/10 | (50%) |
| Extremities | 11/20 | (55%) | 16/26 | (61%) | 5/10 | (50%) |
| Acral | 2/20 | (10%) | 1/26 | (4%) | 0/10 | (0%) |
| Head/neck | 0/20 | (0%) | 2/26 | (8%) | 0/10 | (0%) |

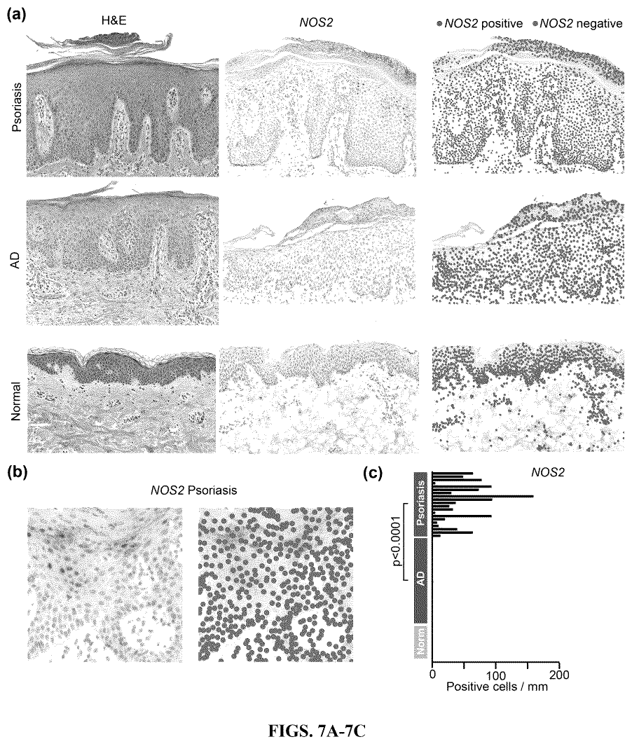

We first evaluated whether RISH staining for NOS2 varied among psoriasis and AD, as would be predicted from analysis of the RNA-seq data. Remarkably, we found that NOS2 staining was present in all 20 psoriasis cases, and that only 1 of 26 cases of AD had any detectable NOS2 expression, and this expression was minimal (FIGS. 7A-7C). No staining was observed in any of the normal controls. NOS2 staining in psoriasis was positive in keratinocytes in the upper stratum spinosum (FIG. 2B). The apparent specificity of the NOS2 with little background emphasized the potential power of RISH as an approach and also provided molecular immunologic validation of the clinicopathologic classifications for cases included in this series.

IL17A and IL13 Staining Patterns Also Differentiate Psoriasis from AD

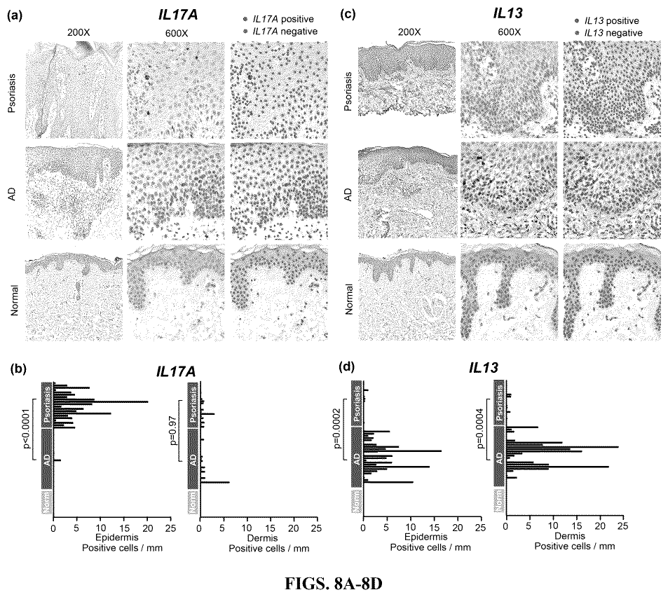

Analysis of the RNA-seq data suggested that IL17A and IL13 might have the best ability to differentially label psoriasis and AD. Further, IL-17A and IL-13 are key targets of treatment in these disorders. We therefore stained cases using RISH probes specific for IL17A and IL13. We found that all 20 cases of psoriasis showed detectable IL17A expression in the epidermis. (FIGS. 8A-8B). Interestingly there were significantly fewer IL17A positive cells in the dermis, despite the majority of the inflammation being present in the dermis. As with the RNA-seq data, we found that there was considerable variability in the relative abundance of IL17A among psoriasis cases. In contrast, only 2 of 26 cases of AD had any detectable IL17A positive cells in the epidermis, and staining was minimal. This may support previous literature suggesting IL-17 may play a driver role in a small subset of AD patients. IL17A staining was not observed in any of the normal controls.

IL13 staining was observed in 85% of the AD cases (FIGS. 8C-8D). While some cases had epidermal predominant IL13, other cases had dermal predominant or exclusive expression (FIG. 3c, 3d). The abundance of IL13 positive cells was also quite variable in the AD cases.

Most cases of psoriasis were negative for IL13; however focal staining was observed in some cases. No IL13 staining was observed in normal skin. Overall IL17A and IL13 staining patterns alone were sufficient to distinguish between psoriasis (IL17A-predominant), AD (IL13-predominant) and normal (negative staining) in of 52 of the 56 cases in the series.

### IL17A and IL13 RISH Staining is Found Predominantly in CD3+ T Cells

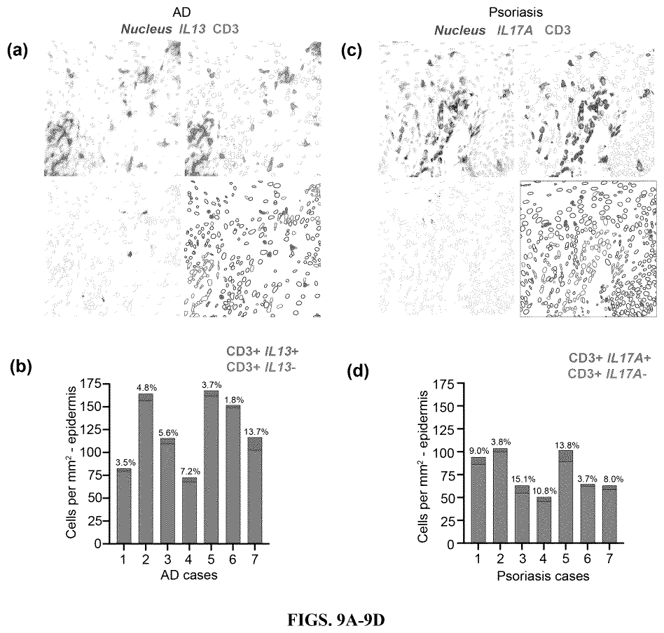

In both IL17A and IL13 RISH stains, cells that stained positively tended to have a lymphocyte morphology as would be expected. In order to explore this and also to further validate the specificity of the approach, we performed double staining for CD3 (IHC) and either IL17A or IL13 in 7 randomly selected cases each of psoriasis and AD. We found that the vast majority of IL17A and IL13 positive cells also co-labeled with CD3, suggesting they may represent type 17 and type 2 polarized T cells, respectively (FIGS. 9A and 9C). We focused on the relative abundance of intra-epidermal cytokine producing T cells as a function of total T cells in the epidermis, as particularly in psoriasis most cytokine producing cells were found here. This analysis showed that cytokine producing T cells ranged from 3.5% to 15.1% of all intra-epidermal T cells in these samples (FIGS. 9B and 9D).

IL17F Levels Generally Correlate with IL17A Levels in Psoriasis

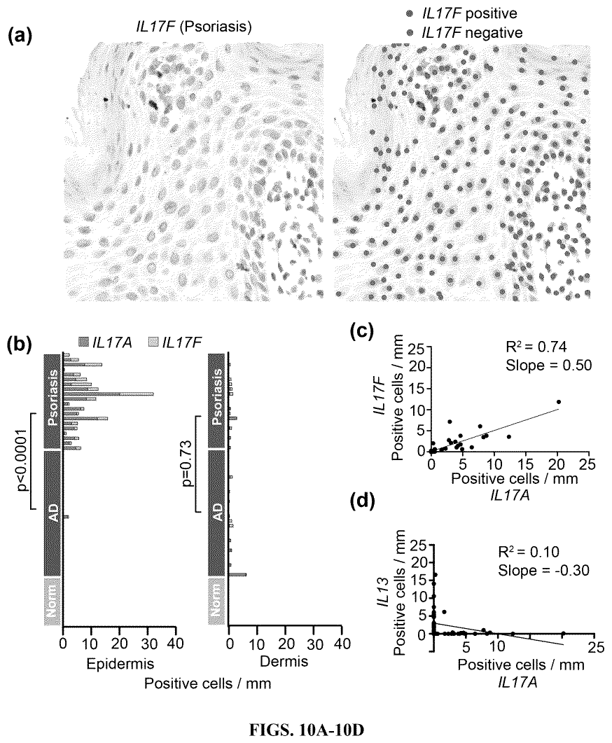

IL-17F is highly homologous to, is often co-expressed with, and can act synergistically with IL-17A. It is conceivable that some cases of psoriasis might be relatively more dependent on IL-17F (or IL17-C) given the observation that some patients respond better to IL-17R blockade than to IL-17A blockade. We next investigated expression of IL17F in this cohort and compared this to IL17A expression. We found that IL17F was detectable in 19 cases of psoriasis, and similarly to IL17A were generally present in the epidermis (FIGS. 10A and 10B). As expected, IL17A tended to be the predominant cytokine in most cases of psoriasis and was highly correlated with IL17F, whereas it was inversely correlated with IL13 (FIGS. 10C and 10D). Interestingly, however, in 3 cases of psoriasis, IL17F was predominant. There was either no or negligible IL17F expression in most cases of AD and no expression was observed in healthy controls.

IL4, IL22, IL31, IL12B (IL-12/23 p40), and IL23A (IL-23 p19) Staining can Also be Used to Characterize Psoriasis and AD

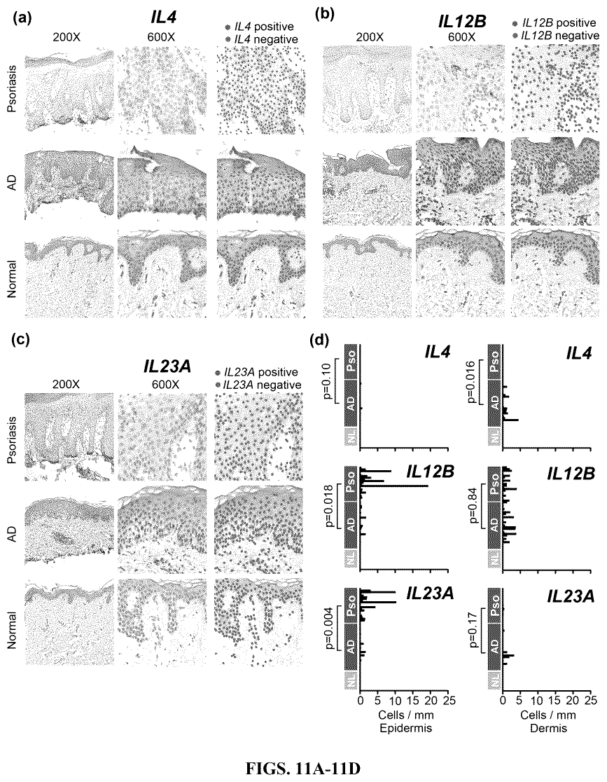

We next used RISH probes for IL4, IL12B, and IL23A to further characterize the skin biopsies from these patients and assess what additional information they might provide, given there are approved drugs that target these cytokines. We found that IL4 expression was much lower than IL13 and was not detected at significant levels in the epidermis of most cases of AD (FIGS. 11A and 11D). The predominance of IL13 over IL4 in AD lesional skin is consistent with prior reports. Expression of IL4 in some cases, but not others, is interesting and might be important in the setting of IL-13 specific inhibition, which is being evaluated in AD.

IL12B (IL-12/23 p40) staining within the epidermis was significantly higher in psoriasis than in AD; however, in the dermis, staining was present in both (FIGS. 11B and 11D). The significance of IL12B staining in the dermis of some of the AD cases is unclear and may represent bystander cells, or cells producing but not secreting IL-12/23. Of note, we also observed IL12B expression in some AD cases in the bulk RNA-seq experiments (FIG. 6B). Interestingly, IL-12/23 (p40) expression has been previously reported in AD, especially in chronic cases and rarely AD patients can improve with ustekinumab. No staining for IL12B was observed in the normal controls. IL23A (IL-23p19) staining within the epidermis was present in most of the psoriasis cases and rarely present in AD cases (FIGS. 11C and 11D). Relatively less staining for IL23A was present in the dermis of both conditions. We were struck that most of the detectable IL23A production using this approach in psoriasis was in the epidermis, as opposed to the dermis. No staining for IL23A was observed in the normal controls.

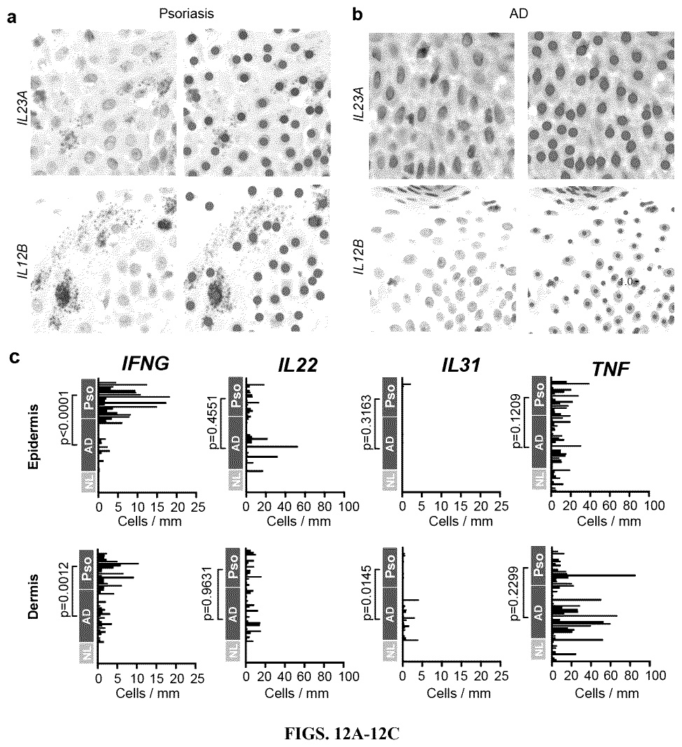

Interestingly, the predominance of staining for both IL12B and IL23A in psoriasis was within the epidermis. Whereas clusters of positive cells predominated in psoriasis, only individual positive cells were present in positive cases of AD (FIGS. 12A and 12B). Although in psoriasis IL-12 and IL-23 have been reported to be predominantly produced by dendritic cells (DCs) in the dermis, production by DCs that either enter the epidermis or reside in the epidermis (e.g. Langerhans cells) in psoriasis has also been described.

We also stained the cases using probes for IL22, IL31, IFNG (IFN-γ), and TNF (TNF-α). As expected, IFNG staining was increased in psoriasis relative to AD (FIG. 12C). IL-31 and IL-22 are emerging treatment targets in AD. Although IL-22 inhibition in AD has been overall less effective than hoped, cases with significant IL-22 expression appear to respond more optimally to IL-22 blockade. IL-22 has also been implicated in psoriasis and may be a cytokine associated with epithelial hyperplasia during chronic inflammation. We found that IL22 was slightly higher in AD cases but was not significantly different between psoriasis and AD (FIG. 12C). IL31 was significantly upregulated in AD compared with psoriasis; most expression was present in the dermis (FIG. 12C).

TNF staining was not able to distinguish AD and psoriasis (FIG. 12C). Analysis of bulk RNA-seq data (FIG. 6A), also showed this. This has been described previously and may relate to the observation that TNF-α mRNA is preproduced intracellularly and held under translational repression and so measurement of mRNA levels alone may not accurately estimate TNF-α activity.

Analysis of Cytokine Expression Patterns with Single Cell RNA Sequencing

Overall, with the RISH staining, we were struck by the epidermal predominant expression of Type 17 cytokines, including IL17A, IL17F, IL12B, and IL23A in psoriasis. To further evaluate this finding, we turned to a recently published single cell RNA sequencing (scRNA-seq) study of psoriasis and AD by Reynolds et al (Reynolds G, Vegh P, Fletcher J, Poyner E F M, Stephenson E, Goh I, et al. Developmental cell programs are co-opted in inflammatory skin disease. Science 2021; 371 (6527).). In this study, the authors used scRNA-seq to compare psoriasis (n=3), AD (n=4) and healthy skin (n=5). Prior to dissociation for scRNA-seq, the epidermis and dermis were separated from each case and analyzed distinct samples. Thus, this provided an ideal data set in which to validate epidermal versus dermal cytokine mRNA expression patterns as determined by RISH, as well as cell-type specificity of expression.

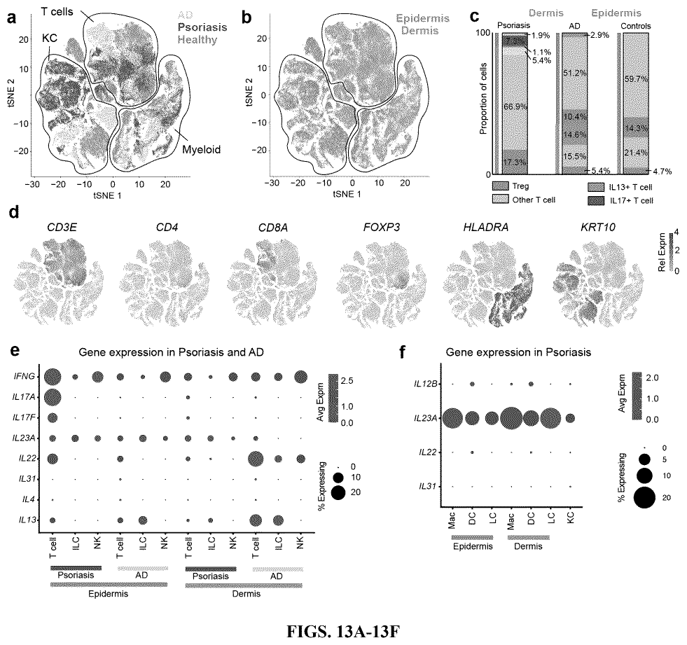

There were 528,253 cells in the Reynold et al study. We focused only on T cells, innate lymphoid cells (ILCs), natural killer cells (NK), other myeloid cells, and keratinocytes according to the cell-type designations as determined by the original authors. Also, we only analyzed data from lesional skin, resulting in a total of 127,219 cells for analysis. Clustering of these data was visualized using t-distributed Stochastic Neighbor Embedding (tSNE) (FIG. 8a, b, and d). Next, we looked at the proportion of T cells in the epidermal versus dermal preparations across samples. In psoriasis, while most T cells were located in the dermis, the vast majority of IL17A producing T cells were found in the epidermis (FIG. 13C). In contrast, while T cells were more evenly distributed between epidermis and dermis in AD, the majority of IL13 production was in the dermis (although there was some in epidermis too) (FIG. 13C). Overall, these patterns are highly consistent with our RISH data, although we did detect relatively more epidermal IL13 in our samples.

Next, we looked more broadly at cytokine expression among T cells, NK cells, ILCs, macrophages, dendritic cells, and keratinocytes, as a function of epidermal versus dermal derivation (FIG. 13E). Most cytokine expression was from T cells, consistent with our observations, but some was also found in ILCs and NK cells. In psoriasis, the majority of IL12B and IL23A expression was from myeloid cells (FIG. 13F). DCs in the epidermis produced the highest levels of IL12B in psoriasis. While the magnitude of IL23A expression was higher in the epidermis, the proportion of cells producing IL23A was slightly higher in the dermis.

Principal Component Analysis Using RISH Patterns Distinguishes Psoriasis from AD

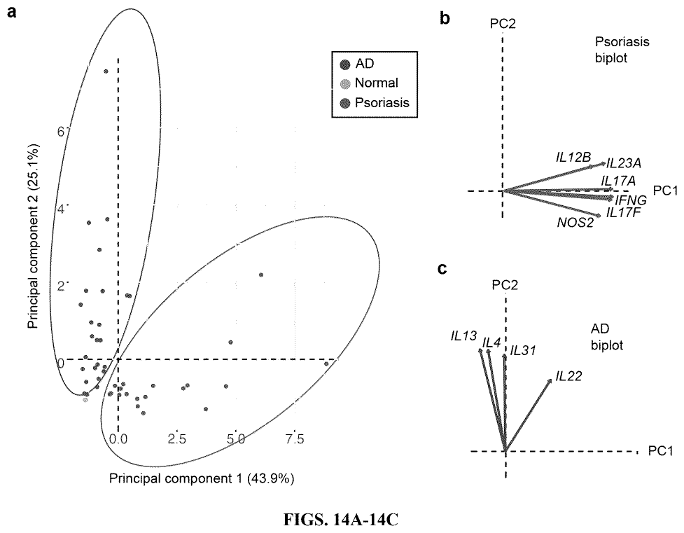

Last, we performed principal component analysis (PCA) on these cases using the RISH cytokine and NOS2 staining data. This analysis showed that that psoriasis cases generally clustered together and AD cases also generally clustered together (FIGS. 14A-14C). Biplot analysis of the PCA plots showed that IL13, IL4, IL31, and IL22 were drivers of clustering along the AD principal component (PC2) whereas NOS2, IL17A, IL17F, IFNG, IL23A, and IL12B were drivers of clustering along the psoriasis component (PC1).

In the past several years there has been a revolution in the molecularly directed treatment of inflammatory skin diseases, including psoriasis and AD. However, clinically implementable and actionable molecular diagnostics in this area have lagged behind, particularly as they relate to personalized treatment selection. A trial-and-error approach to biologic treatment remains standard-of-care; however, such an approach is 1) inefficient, 2) expensive, 3) potentially anxiety-inducing to patients as treatment outcomes are unpredictable, and 4) sometimes accompanied by avoidable adverse effects. Also, some patients may remain on a medication to which they have a suboptimal response when a better alternative in a different class may be available (e.g. IL-12/23p40 versus IL-17A versus IL-23 inhibitor in psoriasis).

Here we show that RISH for disease-causing cytokines is a specific, feasible approach that can identify pathologic cytokines in psoriasis and AD, with IL17A and IL13 appearing to provide the most information. RISH is also an approach that can be easily and cost-efficiently implemented in dermatopathology laboratories, as its workflow is analogous to IHC. The results are also rapid, making it conceivable that data obtained through RISH-based analyses could be incorporated into real-time clinical decision making. In contrast, RNA-seq is expensive, requires specialized tissue collection and processing (including data normalization) and can take weeks, or more.

We observed that there is variability within both psoriasis and AD in terms of the predominant druggable cytokine expressed. In psoriasis, most cases were IL17A-predominant, but others were instead IL17F, IL23A, and/or IL12B predominant. As compared with AD cases, psoriasis cases demonstrated relatively less molecular immunologic heterogeneity, consistent with prior observations and with the excellent responses observed with IL-17 and IL-23 inhibitors in most patients with psoriasis. How this molecular heterogeneity relates to differences in response to treatment and potentially to the development of paradoxical eruptions when a particular cytokine is inhibited will be an area of significant interest moving forward.

Interestingly, most IL-17 mRNA expression appeared to be within the epidermis in psoriasis, a pattern observed both in the RISH staining as well as the scRNA-seq data. These findings were somewhat surprising as they are not necessarily in agreement with prior studies looking at cytokine expression patterns in psoriasis in tissue sections which have reported mostly dermal expression of these cytokines using IHC and/or immunofluorescence (IF). Additional study will be needed to reconcile these observations which may relate to differences in technique and detection of mRNA vs protein.

We also found that, somewhat surprisingly, that most of the IL12B and IL23A production in psoriasis, as detected by RISH, was within the epidermis compared to the dermis. In the scRNA-seq data set IL12B expression was predominantly epidermal, whereas IL23A was produced in both the epidermis and dermis. Prior studies have also found expression of these cytokines by DCs that either enter the epidermis or reside in the epidermis, while other studies have found mostly dermal expression. There was negligible expression by keratinocytes. As with IL-17, additional study will be needed to reconcile differences in location of cytokine production observed in different studies with different techniques.

In AD, while most cases were IL13 predominant, some cases of AD did not show significant expression of either IL13 or IL4, and one case was IL17F predominant. Further, there was relatively more or less IL22 and IL31 staining in individual cases. Of note, in occasional cases IL13 was not detected at significant levels (or at all); whether this represents biology or instead a technical or sampling limitation remains to be determined.

There is thought to be considerable clinical heterogeneity in AD, and response to a targeted inhibition, such as to IL-4Ra blockade, can vary. Some groups have even divided AD into immunologic endotypes; given the observation that IL-17 may play a more prominent role in children with AD and AD patients of Asian descent and that IL-22 may play a more prominent role in some African Americans with AD. Overall, RISH may provide a practical technique to dissect this molecular heterogeneity in a quantifiable and practical fashion. We hypothesize that patients with purer IL13/IL4 dysregulation may respond best to blockade of this axis with dupilumab, while those with mixed or other immunologic drivers may respond sub-optimally and may theoretically respond better to blockade of other cytokines or to more broadly acting cytokine blockers, such as Janus kinase (JAK) inhibitors. JAK inhibitors have the ability to simultaneously inhibit mixed immune response, e.g. Th2 (IL-4, IL-13) and Th17 (IL-12, IL-23). In various embodiments, this approach is used to identify individuals with mixed immune polarization in whom JAK inhibition would be the most appropriate treatment choice (as opposed to a monoclonal antibody).

This study demonstrates the feasibility of RISH for detection of cytokines in inflammatory skin disease. RISH for cytokines could be readily expanded to additional cytokine targets and further applied to these and other inflammatory skin diseases. Minimal background staining is observed with the approach making it very straightforward to quantify and suggesting that reproducibility among different labs is likely to be high. Overall, we predict this approach will be a useful and efficient tool to molecularly phenotype inflammatory skin diseases with implications for both research and clinical care, including personalized treatment selection.

### Example 3

### Study Population

We will enroll up to 40 patients with eczema (20) and psoriasis (20), including some that may have overlapping or atypical features. This is a generally healthy population in the outpatient setting. Patients will be recruited from Dermatology clinics. The patients will either be told of the study during their routine appointment or be contacted by their physician via phone.

### Eligibility Criteria/Vulnerable Populations

Eligibility will be determined through clinical physical examination and through medical record review. Patients that have a presentation consistent with psoriasis or eczematous dermatitis will be determined eligible, presuming they meet other eligibility criteria.

### Study Procedures

Patients seen at the Dermatology practice who are planning to undertake biologic treatment for eczema and psoriasis as part of their clinical care, and who are judged potentially eligible, will be asked to participate.

We will enroll up to 40 patients with eczema (20) and psoriasis (20), including some that may have overlapping or atypical features.

Shave Biopsy: In about 75% of the patients, a shave biopsy would be clinically indicated (SOC) prior to initiation of biologic therapy. A typical shave biopsy is approximately a 1.0 cm×1.0 cm×0.1 cm disc. This biopsy tissue would be sufficient to perform the proposed laboratory analyses. In some instances, two shave biopsies may be performed to improve diagnostic accuracy (standard of care).

In approximately 25% of the patients, the clinician would not typically perform a biopsy, but would treat based on the clinical diagnosis. In this scenario the biopsy would be a research specific procedure. In these patients, a single research biopsy will be obtained. The biopsy size would also be approximately a 1.0 cm×1.0 cm×0.1 cm disc. If a biopsy of the same eruption has been performed in the last 5 years for diagnosis, this biopsy tissue can be utilized instead.

Once the participant's clinical therapy has begun, they may be asked to complete another shave biopsy after 3 months of treatment (at the discretion of the investigator). This biopsy is not required for participation (optional). The biopsy size would also be approximately a 1.0 cm×1.0 cm×0.1 cm disc. The study will record related clinical information as part of the research.

The biologic therapy is selected as part of routine clinical practice for each patient. Participation in the study is fully optional and patients can opt out of the study. RISH will be performed for cytokines IL4, IL12A, IL12B, IL13, IL17A, IL17F, IL22, IL31, IL33, IFNG and TSLP. Measurement of these cytokines using this approach is of course a variation from standard care.

The severity of disease for each patient will be evaluated at baseline before starting biologic therapy using both the v-IGA-AD for eczema11, IGA for psoriasis 12, and BSA affected. Itch will be evaluated using peak pruritus NRS in AD patients13. These are all standard clinical outcomes measures in these diseases and are calculated based on physical examination of the skin and they will be recorded for the research. Photography of the affected areas of skin will be performed. If the participant prefers that particular areas of their skin are not photographed, then they will not be. The photographs will be used by the research team to evaluate changes in the skin over the course of the study. Non-identifiable images may be used for publication and education. Identifiable images will not be used without permission of the participant.

As part of their standard clinical care, the participants will initiate their biologic therapy at the FDA approved dose (standard of care). The agent will be selected according to the standard of care, which incorporates patient medical history, insurance coverage, and physician preference. There is no variation from standard care in treatment selection and the research portions of the study will not influence this choice of agents.

Research biopsies will be used to determine the density of positive cells in the epidermis, density of positive cells in the dermis, mean signal intensity, and mean signal area will be determined for each cytokine in each case. Quantification of scanned slides will be performed using QuPath, an open source quantitative pathology program. Slides will be scanned.

Recording subjects clinical follow up: Patients are seen after 6 weeks and after 3 months of biologic therapy, this is standard of care and no separate visits are required for research. The v-IGA-AD, 5 point IGA for psoriasis for and BSA will be evaluated so that the change in score can be determined for each patient. Change in peak pruritus NRS will also be assessed at both time points and the data recorded for the research.

Three-month Biopsy (not required for participation): An optional biopsy may be performed after 3 months of treatment at the discretion of the investigator and the patient. This is not standard of care. The data may be helpful in further understanding why patients may respond sub-optimally to therapy.

### Data Collection

### Data Will Include:

Patient name, Date of Birth, medical record number,
Patient sex, race, medical comorbidities and medications
Years since diagnosis of psoriasis or eczema
Prior therapies and whether or not they were effective
Therapy which is initiated will be recorded
IGA-AD and/or IGA, BSA involvement, NRS score, and clinical photographs for each clinical visit
RISH cytokine profiles
Other data collected during the clinical visits

### Results:

Patients will show greater responsiveness to biologic therapy targeting cytokines that have been indicated to be at elevated levels by RISH.

The disclosures of each and every patent, patent application, and publication cited herein are hereby incorporated herein by reference in their entirety.

While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of treating an autoimmune or autoinflammatory dermatological condition in a subject in need thereof, the method comprising:
performing RNA in situ hybridization on a tissue sample obtained from the patient with at least one probe that binds to a polyribonucleotide encoding at least one cytokine to determine a level of the at least one cytokine;
comparing the level of the at least one cytokine to the level of the same cytokine in a normal control;
determining that the level of the at least one cytokine deviates from the level of the same cytokine in the normal control; and
providing treatment for the dermatological condition to the subject.

2. The method according to claim 1, wherein the tissue sample is a formalin-fixed paraffin-embedded tissue sample.

3. The method according to claim 1, wherein the tissue sample is a diagnostic biopsy sample.

4. The method according to claim 1, where the cytokine is selected from the group consisting of IL12B, IL17A, IL17C, IL17F, IL23A, IL4 and IL13.

5. The method according to claim 1, where the probe binds IL-12 p40, IL-17A, IL-23 p19, IL-4 or IL-13.

6. The method according to claim 1, wherein the cytokine is selected from the group consisting of IL31, IL33, TSLP, IL12A, IL22, IL36A and IL36G.

7. The method according to claim 1, wherein the method further comprises measuring a level of NOS2.

8. The method according to claim 1, wherein the treatment comprises a monoclonal blocking antibody targeting the at least one cytokine.

9. The method according to claim 1, wherein the dermatological condition is psoriasis and the treatment is ustekinumab.

10. The method according to claim 1, wherein the dermatological condition is atopic dermatitis and the treatment is dupilumab.

11. The method according to claim 1, wherein the RNA in situ hybridization comprises RNAscope analysis.

12. The method according to claim 1, wherein the at least one cytokine comprises a panel of three to five cytokines.
